(19) United States
(12) Patent Application Publication
(10) Pub. No.: US 2005/0080325 A1
(43) Pub. Date: Apr. 14, 2005

(54) Low profile connector and system for implantable medical device

(75) Inventor: John H. Erickson, Plano, TX (US)

Correspondence Address: DOCKET CLERK, DM/ANSI, P.O. BOX 802432, DALLAS, TX, 75380, US

(73) Assignee: Advanced Neuromodulation Systems, Inc., Plano, TX (US)

(21) Appl. No.: 10/965,530

(22) Filed: Oct. 14, 2004

Related U.S. Application Data
(60) Provisional application No. 60/511,717, filed on Oct. 14, 2003.

(51) Int. Cl. A61N 1/05

(52) U.S. Cl. 600/395; 606/151

(57) ABSTRACT

An implantable medical device has a portion formed from a shape memory alloy (SMA) and adapted to connect to another device. At a lower temperature, the SMA is deformed such that the two devices may be mated with low insertion force. At a higher temperature, e.g., the internal temperature of the human body, the SMA attempts to return to its original shape, creating a connection between the two devices and causing a retention force that resists disconnection of the two devices.

### TECHNICAL FIELD OF THE INVENTION

The present invention relates to connectors, and in particular, a connector for use with implantable medical devices.

### BACKGROUND

Implantable leads having electrodes are used in a variety of applications; including the delivery of electrical stimulation to surrounding tissue, neural or otherwise, as well as measuring electrical energy produce by such tissue. Some leads include lumens (or channels) for the delivery of other elements, including chemicals and drugs. Whether in a stimulation, sensing or element delivery capacity, such leads are commonly implanted along peripheral nerves, within the epidural or intrathecal space of the spinal column, and around the heart, brain, or other organs or tissue of a patient.

Generally, several elements (conductors, electrodes and insulation) are combined to produce a lead body. A lead typically includes one or more conductors extending the length of the lead body from a distal end to a proximal end of the lead. The conductors electrically connect one or more electrodes at the distal end to one or more connectors at the proximal end of the lead. The electrodes are designed to form an electrical connection or stimulus point with tissue or organs. Lead connectors (sometimes referred to as contacts, or contact electrodes) are adapted to electrically and mechanically connect leads to implantable pulse generators or RF receivers (stimulation sources), or other medical devices. An insulating material typically forms the lead body and surrounds the conductors for electrical isolation between the conductors and for protection from the external contact and compatibility with a body.

Such leads are typically implanted into a body at an insertion site and extend from the implant site to the stimulation site (area of placement of the electrodes). The implant site is typically a subcutaneous pocket that receives and houses the pulse generator or receiver (providing a stimulation source) . The implant site is usually positioned a distance away from the stimulation site, such as near the buttocks or other place in the torso area. In most cases, the implant site (and insertion site) is located in the lower back area, and the leads may extend through the epidural space (or other space) in the spine to the stimulation site (middle or upper back, or neck or brain areas).

The process of implanting medical treatment devices in the body of a patient typically proceeds in at least two steps. First, one or more leads are implanted by passing the lead through an insertion needle to reach the stimulation site or by surgical emplacement of the lead. Second, a medical device is connected to the lead or leads and placed in the implant site. Leads may be connected in series to reach a treatment location that is at a greater distance from the subcutaneous pocket than can be reached with a single lead.

Many leads used to deliver treatment have a small cross section. This facilitates their implantation in the body and minimizes the unwanted side effects of their implantation. As a result of their smaller cross section, these leads are more fragile and less resistant to the forces exerted upon them during the process of connecting them to another implantable medical device.

The connection between an implantable lead and an implantable medical device (which may be another implantable lead or an implantable treatment device) typically employs either springs or setscrews to apply force to the lead for several purposes. One purpose is to provide a retention force to maintain the connection against external forces that might separate the lead from the device. Another purpose is to provide a contact force to make an electrical connection between contacts in the device and a connector on an electrical lead. Yet another purpose is to provide a contact force to create a seal around a lead with a lumen.

As a lead is inserted into a connector with springs, the lead generally supplies a force to displace the spring-loaded contacts or seals. Leads of smaller cross section may not be able to supply this insertion force without suffering damage. A connector employing setscrews may require less insertion force, however the torque applied to the setscrews is generally limited in order to avoid stripping the setscrews, twisting the connector block, or crushing or deforming the lead.

Additionally, setscrews and springs typically lie adjacent to the longitudinal axis of a lead, in order to apply forces perpendicular to that axis. As a result, the cross section of the connector must be large enough in at least one dimension to encompass the diameter of the lead and to provide space for the spring or setscrew. Such a connector is referred to as a high profile connector.

Many other problems and disadvantages of the prior art will become apparent to one skilled in the art after comparing such prior art with the present invention as described herein.

### SUMMARY

The present invention provides a low profile connector for implantable medical devices with low insertion force, high retention force, and a reduced likelihood of lead damage during the formation of a connection.

More specifically, aspects of the invention can be found in an implantable medical device having a portion for connecting to another device. The portion includes a shape memory alloy.

Other aspects of the invention may be found in an implantable connection system including two implantable devices. Portions of the devices are adapted to connect together and one of the portions includes a shape memory alloy.

Aspects of the invention can also be found in an implantable system for stimulating a portion of the body. The system has a stimulation source and a lead for delivering the stimulation from the source to the portion of the body being stimulated. The lead and the source are adapted to connect together and one of the lead and the source has a portion formed from a shape memory alloy.

Yet other aspects of the present invention can be found in a method of connecting implantable medical devices. The method includes inserting a portion of an implantable medical device into a portion of another implantable medical device. The two portions are adapted to connect together and one of the portions includes a shape memory alloy. The method further includes increasing the temperature of the two portions above the transformation temperature to create a connection between the two medical devices.

Aspects of the invention can also be found in a method of manufacturing an implantable medical device. The method includes providing an implantable medical device that has a portion adapted to connect to another device. The method further includes constructing the portion of the device from a material that includes a shape memory alloy.

### BRIEF DESCRIPTION OF THE DRAWINGS

For a more complete understanding of the present invention, and the advantages thereof, reference is now made to the following descriptions taken in conjunction with the accompanying drawings, wherein like numbers designate like objects, and in which:

FIG. 1 is a cutaway view of an implantable electrical stimulation device and lead employing an embodiment of the invention;

FIG. 2 is a cutaway view of a lead-to-lead connection embodying the present invention;

FIG. 3 is an orthogonal view of an implantable infusion pump and lead according to the invention;

FIGS. 4a and 4b are orthogonal views of a connection according to the invention;

FIGS. 5a and 5b are orthogonal views of a spiral spring embodiment of the invention;

FIG. 6 is an orthogonal view of an embodiment of the present invention employed to clamp a connector;

FIG. 7 is a flow chart of a process of connecting and implanting medical devices according to the present invention;

FIG. 8 is a cutaway view of another embodiment of the stimulation device and lead in accordance with the present invention; and

FIG. 9 is another embodiment of a connection in accordance with the present invention.

### DETAILED DESCRIPTION OF THE INVENTION

Shape memory alloys (SMAs) are materials that can return to a predetermined shape when heated or cooled. While above its transformation temperature, a SMA is capable of being formed into an original shape by certain metal-working techniques, among them, extrusion, forging, hot rolling, and forming. The SMA enters another state when cooled below its transformation temperature; in this state the SMA is capable of deformation. SMAs retain a deformed shape until heated above the transformation temperature, whereupon a change in crystal structure causes the SMA to return to its original shape. One attribute of SMAs is the ability to generate extremely large recovery stresses, i.e., exerting large forces, when constrained from returning to its original conformation. One example of a SMA is a nickel-titanium alloy called NiTiNOL. Other examples include copper-aluminum-nickel, copper-zinc-aluminum, and iron-manganese-silicon alloys.

The transformation temperature of a SMA is determined by the ratio of its alloy constituents. NiTiNOL with a composition of approximately 55.6 percent nickel by weight has a transformation temperature in the range of 20° to 40° C. (68° to 104° F.), the exact transformation temperature being determined by the actual composition of the alloy. NiTiNOL with 55.1 to 55.5 percent nickel by weight has a transformation temperature in the range of 45° to 95° C. (104° to 203° F.). NiTiNOL with about 55.8 percent nickel by weight has a transformation temperature in the range of 10° to 20° C. (50° to 68° F.). Thus, the desired transformation temperature for an application employing a SMA determines the exact composition of the alloy to be used.

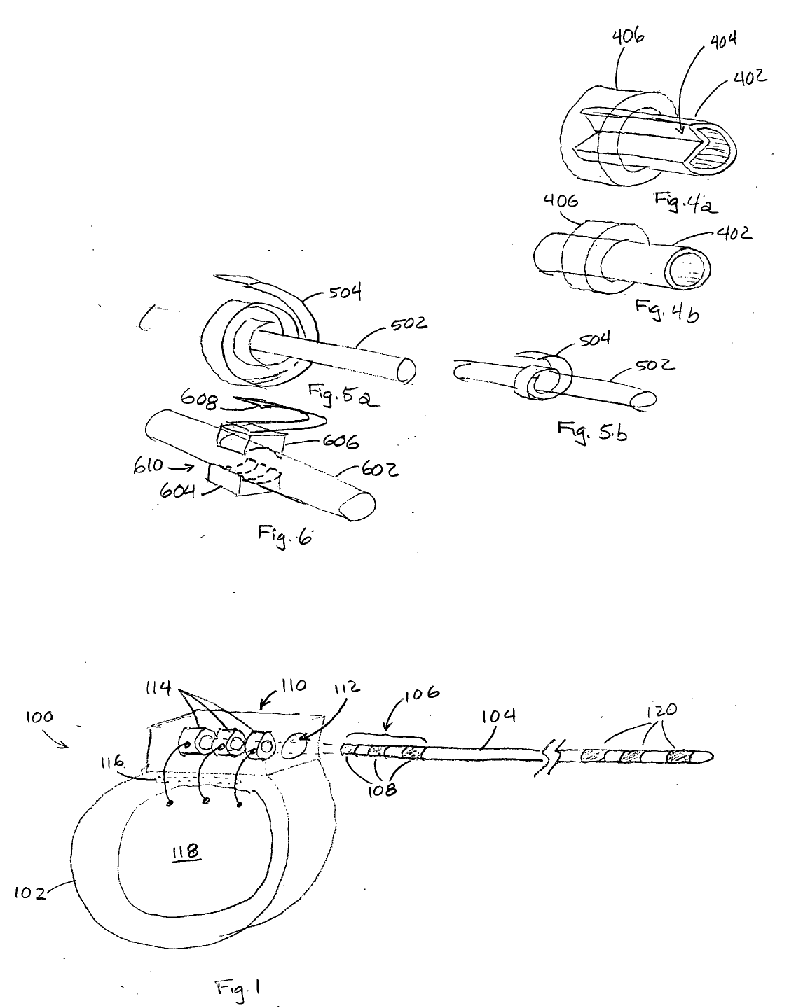

One embodiment of the present invention is shown in FIG. 1. An implantable neurostimulation system 100 includes an implantable pulse generator (IPG) 102 and an implantable stimulation lead 104. Leads of this type are described more fully in U.S. Pat. No. 6,216,045, which is incorporated herein by reference. An exemplary IPG may be one manufactured by Advanced Neuromodulation Systems, Inc., such as the Genesis® System, part numbers 3604, 3608, 3609, and 3644. The lead 104 includes a connector portion 106 with terminals 108 on a proximal end. Electrodes 120 are located on a distal end of the lead 104 and are connected to the terminals 108 by conductors (not shown) within lead 104. To connect the lead 104 to the IPG 102, the connector portion 106 is inserted into a receiving end or header 110 through an opening 112. Contacts 114 are positioned to mate with the terminals 108 of the connector portion 106. The contacts 114 are connected by conductors 116 to the source of the stimulation signals in circuitry 118.

As will be appreciated, while the embodiment shown in FIG. 1 has the individual contacts 114 mating with the individual terminals 108, a single contact could be sized and positioned to mate with more than one terminal or a single terminal could mate with more than one contact without departing from the spirit and scope of the invention.

The contacts 114 are constructed to include a shape memory alloy (SMA) material. As shown in FIG. 1, the contacts 114 are in a first state, at a temperature below the transformation temperature of the SMA material, and the SMA material has been deformed to have an inner diameter of a size sufficient to accept the connector portion 106 with a low insertion force. In a second state of the contacts 114, the original shape of the SMA material is similar to the deformed shape, but has an inner diameter of a size the same as or smaller than the outer diameter of terminals 108.

After the insertion of the connector portion 106 into the receiving end 110, the temperature of the contacts 114 is increased to a temperature above the transformation temperature of the SMA. This causes the SMA material to attempt to return to its original shape, thereby contracting around the terminals 108. As the inner diameter of the contacts 114 decreases and they touch the terminals, any further change in the crystal structure of the SMA will cause the contacts to apply force to the terminals. This provides both an electrical contact force between the contacts 114 and the terminals 108, and a retention force (or mechanical contact force) that increases the insertion or removal force of the connector portion 106 and the receiving end 110.

Preferably, the transformation temperature of the SMA from which the contacts 114 are fabricated is chosen to be below the internal body temperature of the human body. In this way, once the implantable system 100 is implanted in a body, the temperature of the contacts 114 will rise above (or, if previously heated, remain above) the transformation temperature of the SMA and the electrical contact and retention forces on the terminals 108 and the connector portion 106 will be maintained. In the embodiment illustrated in FIG. 1, the composition of the shape memory alloy is chosen to result in a transformation temperature of about 85° F. to about 90° F. Other transformation temperatures may be chose consistent with the principles of the present invention.

In the event it is desired to separate the lead 104 and the IPG 102 (for example, to allow replacement of the IPG), the IPG 102 may be accessed with a surgical procedure and the temperature of the contacts 114 lowered below the transformation temperature of the SMA. This can be achieved, for example, by submersing the IPG 102 in an ice bath. Once the contacts 114 are below the transformation temperature, the SMA will return to its deformed shape and the contacts 114 to their first state, whereupon the connector portion 106 of the lead 104 can be removed from the receiving end 110 with lower force.

As will be appreciated, any number of conductors (not shown), electrodes 120 and terminals 108 may be utilized, as desired. For purposes of illustration only, the lead 104 is shown with three terminals 108 and three electrodes 120. It will be further understood that the distal end of the lead 104 is shown with band electrodes 120. Other types, configurations and shapes of electrodes may be utilized as known to those skilled in the art. Likewise, other types, configurations and shapes of terminals (and connector portions) may be used, as desired.

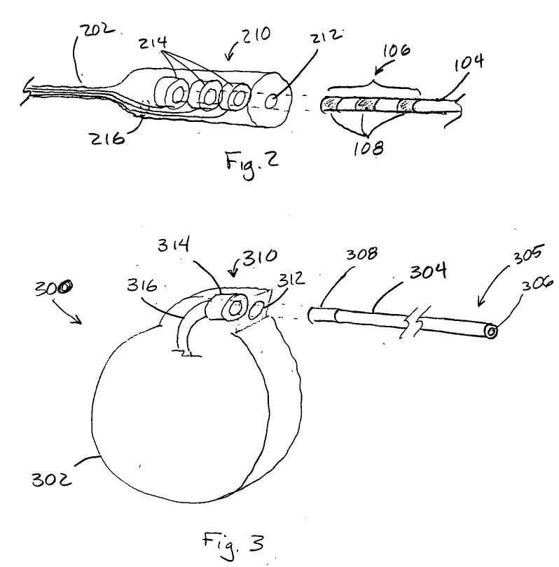

Turning to FIG. 2, another embodiment of the present invention is shown, wherein an electrical stimulation lead is connected to an extension in accordance with the present invention. The implantable stimulation lead 104 (from FIG. 1) is connected to an implantable lead extension 202 by inserting the connector portion 106 into a receiving end 210 through an opening 212. As in the embodiment shown in FIG. 1, contacts 214 mate with the terminals 108, with conductors 216 supplying the stimulation signals to the contacts 214 from a stimulation source (not shown). The extension 202 might terminate at its other end in a connector portion similar to the connector portion 106 of the lead 104, or it might be fabricated as a “pigtail”, permanently attached to a treatment device.

As described for the embodiment shown in FIG. 1, the contacts 214 are constructed to include a SMA material and, at a temperature below the transformation temperature of the SMA, the contacts 214 are deformed to accept the connector portion 106 into the receiving end 210 with a low insertion force. When the contacts 214 are raised above the transformation temperature, by implantation into the body or by heating prior to implantation, the SMA will attempt to resume its original shape, thereby making electrical contact between the contacts 214 and the terminals 108. The connection between the leads 104 and 202 is maintained at the internal temperature of the human body. The electrical contact and retention force between the contacts 214 and the terminals 108 is reduced by lowering the temperature of the contacts 214 below the transformation temperature of the SMA material.

It will be understood by one skilled in the relevant art that it is within the spirit and scope of the invention to utilize a SMA whose transformation temperature is above the internal temperature of the human body. In an embodiment of this aspect of the invention, a connector portion would be inserted into a receiving end while above the transformation temperature of the SMA. The temperature of the connection between the two devices would then be lowered, causing the SMA to attempt to return to its deformed shape, thereby exerting contact and retention forces between the elements of the connector portion and the receiving end. After implantation in the body, the SMA would remain in its deformed shape below its transformation temperature, thereby maintaining the connection between the medical devices.

Yet another embodiment of the invention is shown in FIG. 3, which illustrates an implantable drug treatment system 300, including an implantable treatment device 302 (in this embodiment, an infusion pump) and an implantable delivery lead 304. The lead 304 has a lumen (or passage) 306 running its length to deliver a chemical or drug to a treatment site in the body at the distal end 305 of the lead 304, remote from the treatment device 302. A connector portion 308, at the proximal end of the lead 304, may be inserted into a receiving end 310 of the treatment device 302, where a connector portion 308 mates with a contact 314. The source of the chemical or drug, in this embodiment, is a drug reservoir and pump mechanism within the treatment device 302, which is not shown in FIG. 3. The drug or chemical is conducted from that source to the contact 314 by a tube 316.

As with the connection of the electrical stimulation system of FIG. 1, the contact 314 is preferably constructed to include a SMA material having a transformation temperature below the internal temperature of the body. While the SMA is below that transformation temperature, the contact 314 is in a first state, deformed to have an inner diameter of sufficient size to accept the connector portion 308 with a low insertion force. Once the connector portion 308 and the contact 314 are mated together, the temperature of the contact 314 is raised above the transformation temperature and the SMA attempts to resume its original shape, causing the contact 314 to enter a second state. In this embodiment, too, the contact 314 in its second state has an inner diameter of a size equal to or smaller than the outer diameter of the connector portion 308, resulting in contact between the contact 314 and the connector portion 308. The force exerted by the SMA material seals the contact 314 to the connector portion 308, ensuring that all the chemical or drug pumped by the treatment source flows through the lumen 306 to the distal end 305 of the lead 304, at the treatment site. The force between the contact 314 and the connector portion 308 also provides a retention force to prevent the lead 304 and the treatment device 302 from separating.

In the event it is desired to separate the lead 304 and the treatment device 302, the connection may be broken by lowering the temperature of the contact 314 below the transformation temperature of the SMA. The contact 314 will then resume its deformed shape, thereby reducing the retention force on the connector portion 308 and allowing it to be withdrawn from the receiving end 310.

As will be appreciated, the apparatus and techniques of the embodiment in FIG. 2 may also be used to form a lead-to-lead connector for the implantable delivery lead 304.

Insulating spacers or O-rings (not shown) are positioned between the contacts 114, 214, 314 to isolate and seal each contact from one another. In another embodiment, each contact 114, 214, 314 has a spacer positioned on each lateral side of the contact.

Thus, FIGS. 1-3 show embodiments of the invention that allow an implantable lead to be connected to another implantable medical device. The other device may be an implantable treatment device, such as a pacemaker, neurostimulator, wireless receiver, defibrillator or infusion pump, or another implantable lead. An implantable lead adapted to include both conductors to deliver electrical stimulation and one or more lumens to deliver chemicals or drugs to a treatment site may include aspects of the present invention, employed to connect such a lead to a stimulus and treatment source or to another such lead.

The SMA material used to fabricate connections embodying the present invention may include an outer layer or plating of platinum (not shown) to reduce corrosion and/or increase electrical conductivity between the connectors/contacts, or some other corrosion resistant and/or conductive material(s), or other non-shape memory alloy material. The outer layer generally has a thickness in the range of a few microns to a few thousand microns. In the embodiment shown in the Figures, the contacts (or SMA material) form a direct electrical and mechanical connection with the terminals 108. Also, the terminals 108 may directly electrically connect with the outer layer (described above).

In FIGS. 4-6, further embodiments of the invention are illustrated, showing different techniques for forming the connection between the implantable lead and the other implantable device. FIGS. 4a and 4b illustrate an embodiment in which a connector portion 402 is constructed to include a SMA material. FIG. 4a illustrates that the connector portion 402 is deformed by collapsing a section 404 of the sidewall when the temperature of the connector portion 402 is below the transformation temperature of the SMA. This puts the connector portion 402 into a first state, with a deformed shape having a reduced outer diameter that can be inserted into a contact 406 with a lower insertion force. Above the transformation temperature of the SMA, in a second state of the connector portion 402, the original shape of the connector portion 402 has a circular cross-section. Raising the temperature of the connector portion 402 above the transformation temperature causes the SMA material to attempt to return to the original shape, thereby forming the connection with the contact 406, as shown in FIG. 4b.

Unlike the embodiments shown in FIGS. 1-3, in the embodiment of FIGS. 4a and 4b, the connector portion 402 exerts a force upon the contact 406. However, as in those other embodiments, a contact force is created, resulting in an electrical contact or fluid seal between the connector portion 402 and the contact 406, and a retention force is created which resists the separation of the connector portion 402 and the contact 406. Lowering the temperature of the connector portion 402 below the transformation temperature of the SMA causes the SMA to resume the deformed shape, thereby reducing the contact force and allowing the connector portion 402 to be removed from the contact 406 against a reduced retention force.

The connector portion 402 of FIGS. 4a and 4b may alternatively be fabricated as a tube with sidewalls made of braided SMA wire (not shown). If so fabricated, the tube would be placed in a first state with a reduced outer diameter by twisting, rather than by collapsing the sidewall. When heated, such a tube would enter a second state, having the original outer diameter, by untwisting.

FIGS. 5a and 5b show another embodiment of the present invention wherein a contact 504 is constructed to include a SMA in the shape of a coil spring. In a first, low-temperature, state of the contact 504, the deformed shape of the SMA material is an expanded coil, into which a connector portion 502 can be placed, as shown in FIG. 5a, with a lower insertion force. When the temperature of the contact 504 is raised above the transformation temperature, the SMA returns to an original tightly coiled shape, the second state of the contact 504, thereby connecting the connector portion 502 and the contact 504, as shown in FIG. 5b. When the temperature of the contact 504 is lowered below the transformation temperature, the SMA returns to the first state, shown in FIG. 5a, thereby allowing the removal of the connector portion 502 with a lower force.

FIG. 9 shows another embodiment of the present invention wherein the contacts 114, 214, 314 are constructed to include a SMA in the shape of helical coil or wound wires. Though not shown, in a first, low-temperature, state of the contacts 114, 214, 314, the deformed shape of the SMA material is in expanded form (lower insertion force) . When the temperature of the contacts 114, 214, 314 is raised above the transformation temperature, the SMA returns to an original contracted shape or form (higher insertion force). Other designs or shapes (not shown) may be utilized, such as a beam, a spring, and the like.

In the embodiment shown in FIG. 6, a connector portion 602 is inserted into a contact 610, comprising blocks 604 and 606 and an element 608. The block 604 is attached to the housing of the receiving end into which the connector portion 602 has been inserted. The block 606 is attached to one end of the element 608, which is constructed to include a SMA material. The other end of the element 608 is attached to the housing of the receiving end. In the first state of the contact 610, the element 608 is in a deformed shape wherein the ends of the element 608 are closer together, thereby pulling the block 606 away from the block 604 and allowing the connector portion 602 to be inserted with a lower insertion force.

When the temperature of the contact 610 is raised above the transformation temperature of the SMA, the contact 610 enters a second state. In this state, the ends of the element 608 spread apart, attempting to return to the original shape of the element 608. One end of the element 608 is held in place by the housing of the receiving end, so the attempted separation of the ends of the element 608 forces the block 606 toward the block 604, thereby clamping the connector portion 602 between the blocks 604 and 606 and providing an electrical contact and retention force for the connection. Lowering the temperature of the contact 610 below the transformation temperature of the SMA returns the contact 610 to its first state, bringing the ends of the element 608 closer together, moving the block 606 away from the block 604, and reducing the retention force on the connector portion 602.

For purposes of illustration only, the connector portions and terminals shown in FIGS. 1-6 are round in cross-section. As will be appreciated, other types, configurations, cross-sections and shapes of connectors and terminals may be utilized, as known to those skilled in the art, without departing from the spirit and scope of the invention.

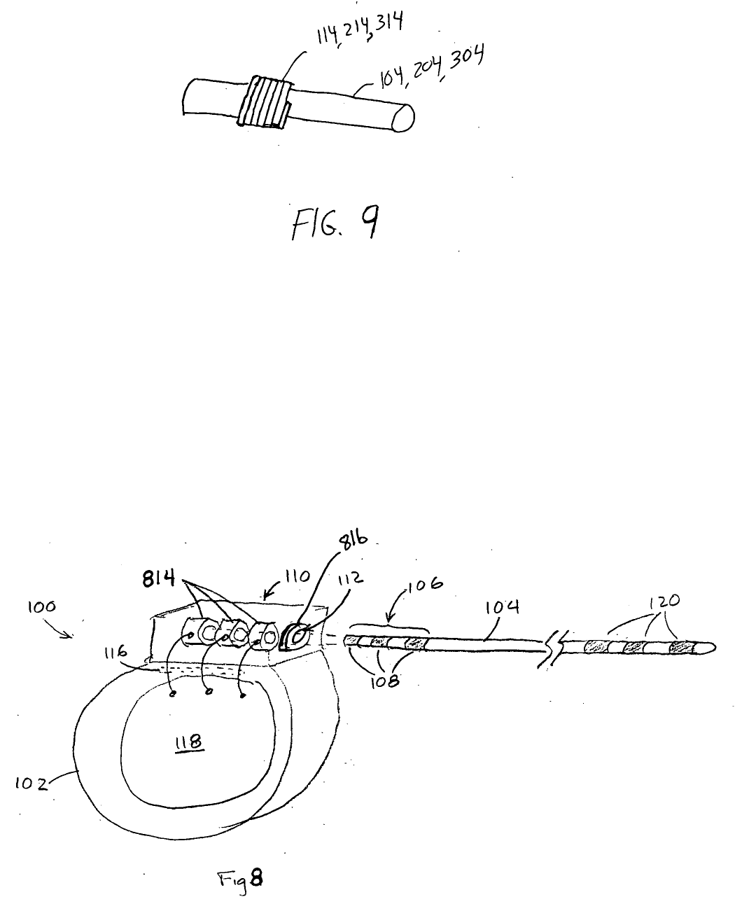

Now turning to FIG. 8, there is shown another embodiment similar to the embodiment shown in FIG. 1. This embodiment is essentially the same as shown in FIG. 1, except that the contacts 814 may or may not include SMA material. A mechanical contact 816 constructed of SMA material is provided that functions in the same manner as described herein, except the contact 816 is utilized to apply a mechanical force that assists in maintaining the insertion of the lead without providing electrical connection to the lead. Some prior art connections utilized one or more set screws that required tightening/loosening with a tool to insert/remove the lead in the connector. In this embodiment, the contact 816 functions similarly to a set screw (tightening/loosening) by increasing the mechanical force when the SMA material is above the transformation temperature. As will be appreciated, this embodiment may also apply to the contacts 214 and 314 in FIGS. 2 and 3 and include the mechanical contact 816.

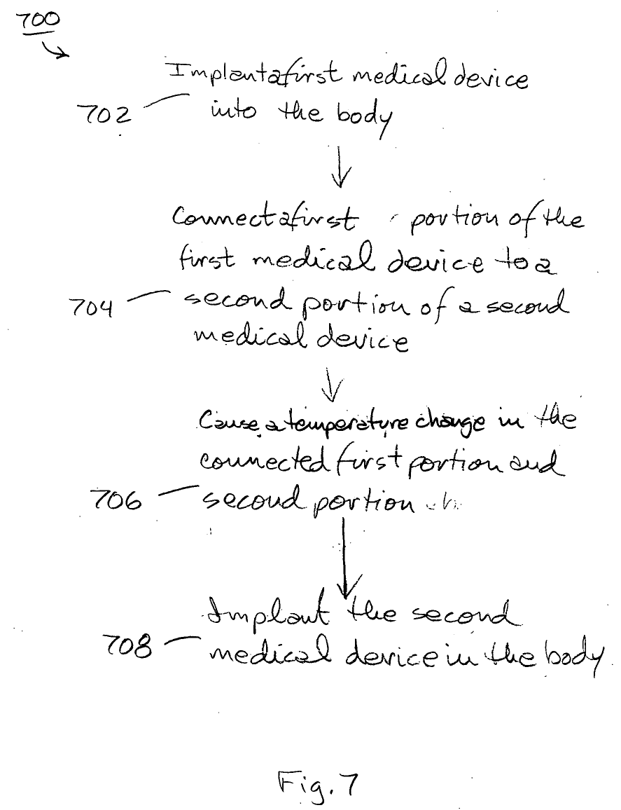

The process of connecting and implanting two medical devices according to the present invention is illustrated in FIG. 7. Process 700 begins with step 702, in which a first medical device, e.g., a stimulation lead, is implanted in the body. Next, in step 704, a first portion of the first medical device is connected to a second portion of a second medical device. For example, the connector portion of a stimulation lead may be inserted into the receiving end of a stimulation device or another stimulation lead. At least one of the first portion and second portion comprises a shape memory alloy (SMA) material, which in this step is in a first state at a first to allow the insertion of one portion into the other portion with a lower insertion force.

In step 706 of the process, the temperature of the connected first portion and second portion is then changed to a second temperature, causing the SMA to enter a second state. In this second state, the SMA attempts to change shape, thereby applying a contact force between the first portion and second portion. A retention force is also created, causing a removal force required to separate the first portion and second portion to be higher than the insertion force. In step 708, the connected first and second medical devices are implanted in the body, where the temperature of the medical devices remains above the transformation temperature of the SMA. It will be appreciated that step 708 may be performed before step 706, wherein the temperature of the connected first portion and second portion is changed to the second temperature by implantation in the body.

It may be advantageous to set forth definitions of certain words and phrases that may be used within this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and if the term “controller” is utilized herein, it means any device, system or part thereof that controls at least one operation, such a device may be implemented in hardware, firmware or software, or some combination of at least two of the same. It should be noted that the functionality associated with any particular controller may be centralized or distributed, whether locally or remotely.

Although the present invention and its advantages have been described in the foregoing detailed description and illustrated in the accompanying drawings, it will be understood by those skilled in the art that the invention is not limited to the embodiment(s) disclosed but is capable of numerous rearrangements, substitutions and modifications without departing from the spirit and scope of the invention as defined by the appended claims.

1. An implantable medical device comprising:
a first portion adapted to provide mechanical and electrical connection to a second device, the first portion comprising, a shape memory alloy.

2. The implantable medical device of claim 1 wherein the implantable medical device comprises a one of an implantable pulse generator and a lead.

3. The implantable medical device of claim 1 wherein the shape memory allow comprises an outer layer of a non-shape memory alloy material.

4. The implantable medical device of claim 1 wherein the shape memory alloy comprises NiTiNOL.

5. The implantable medical device of claim 1 wherein the shape memory alloy is in a first state prior to a connection between the first portion and the second device and in a second state after the connection between the first portion and the second device.

6. The implantable medical device of claim 5 wherein in the first state the shape memory alloy is deformed and in the second state the shape memory alloy is substantially in its original shape.

7. The implantable medical device of claim 5 wherein the shape memory alloy enters the first state at a first temperature and enters the second state at a second temperature.

8. The implantable medical device of claim 7 wherein the first temperature is below the second temperature.

9. The implantable medical device of claim 7 wherein the first temperature is below 90° F. and the second temperature is above 90° F.

10. An implantable connection system, comprising:
a first implantable device having a first portion; and
a second implantable device having a second portion,
wherein the first portion and second portion are adapted to provide mechanical and electrical connection between the first portion and the second portion, and a one of the first portion and second portion comprises a shape memory alloy.

11. The implantable connection system of claim 10 wherein a one of the first implantable device and the second implantable device comprises a lead.

12. The implantable connection system of claim 11 wherein the shape memory alloy comprises an outer layer comprising a non-shape memory alloy material.

13. The implantable connection system of claim 10 wherein the shape memory alloy comprises NiTiNOL.

14. The implantable connection system of claim 10 wherein the shape memory alloy is in a first state prior to a connection between the first portion and the second portion and in a second state after the connection between the first portion and the second portion.

15. The implantable connection system of claim 14 wherein in the first state the shape memory alloy is deformed and in the second state the shape memory alloy is in its original shape.

16. The implantable connection system of claim 14 wherein the shape memory alloy enters the first state at a first temperature and enters the second state at a second temperature.

17. The implantable connection system of claim 16 wherein the first temperature is below the second temperature.

18. The implantable connection system of claim 16 wherein the first temperature is below 90° F. and the second temperature is above 90° F.

19. An implantable system for delivering a stimulus to a portion of the body, comprising:
a source for generating the stimulus and having a first portion; and
a lead for conducting the stimulus from the source to the portion of the body, the lead having a second portion, and wherein the first portion and second portion are adapted to provide mechanical and electrical connection between the first portion and the second portion, and a one of the first portion and second portion comprises a shape memory alloy.

20. The implantable system of claim 19, wherein the shape memory alloy comprises NiTiNOL.

21. The implantable system of claim 19, wherein the shape memory alloy is in a first state prior to a connection between the first portion and the second portion and in a second state after the connection between the first portion and the second portion.

22. The implantable system of claim 21, wherein in the first state the shape memory alloy is deformed and in the second state the shape memory alloy is in its original shape.

23. The implantable system of claim 21, wherein the shape memory alloy enters the first state at a first temperature and enters the second state at a second temperature.

24. The implantable system of claim 23 wherein the first temperature is below the second temperature.

25. The implantable system of claim 23, wherein the first temperature is below 90° F. and the second temperature is above 90° F.

26. A method of connecting implantable medical devices, comprising:
inserting at a first temperature a first portion of a first implantable medical device into a second portion of a second implantable medical device, wherein the first portion and second portion are adapted to connect together and a one of the first and second portions comprises a shape memory alloy; and
causing a temperature change of the shape memory alloy from the first temperature to a second temperature, thereby increasing a contact force between the first portion and second portion.

27. The method in accordance with claim 26, wherein the first temperature is below the second temperature.

28. The method in accordance with claim 26, wherein the first temperature is below 90° F. and the second temperature is above 90° F.

29. The method in accordance with claim 26, wherein the shape memory alloy is in a first state prior to inserting the first portion into the second portion and in a second state after causing the temperature change of the shape memory alloy.

30. The method in accordance with claim 29, wherein in the first state the shape memory alloy is deformed and in the second state the shape memory alloy is in its original shape.

31. A method of manufacturing an implantable medical device, comprising:
providing an implantable medical device having a first portion adapted to connect to a second device; and
constructing the first portion of a material comprising a shape memory alloy.

32. The method according to claim 31, further comprising:
deforming the shape memory alloy while at a temperature below the transformation temperature of the shape memory alloy.

33. A method of implanting medical devices within a human body, comprising:
implanting a first medical device into the body, the first medical device having a first portion;
inserting at a first temperature the first portion into a second portion of a second medical device, wherein a one of the first and second portions comprises a shape memory alloy; and
implanting the second medical device in the body, wherein after implantation of the second medical device the shape memory alloy is at a second temperature, thereby increasing a contact force between the first portion and second portion.

34. The method in accordance with claim 33, wherein the first temperature is below the second temperature.

35. The method in accordance with claim 33, wherein the first temperature is below 90° F. and the second temperature is above 90° F.

36. The method in accordance with claim 33, wherein the shape memory alloy is in a first state prior to inserting the first portion into the second portion and in a second state after implantation of the second medical device.

37. The method in accordance with claim 36, wherein in the first state the shape memory alloy is deformed and in the second state the shape memory alloy is in its original shape.
